(19) United States
(12) Patent Application Publication
(10) Pub. No.: US 2025/0295446 A1
(43) Pub. Date: Sep. 25, 2025

(54) Neuronavigation-Compatible Inner Cannula for Percutaneous Radiofrequency Lesioning

(71) Applicant: University of Cincinnati, Cincinnati, OH (US)

(72) Inventors: Joel Kaye, Cincinnati, OH (US); Jonathan Forbes, Cincinnati, OH (US); Sam Anand, West Chester, OH (US); Manish Raj Aryal, Cincinnati, OH (US)

(73) Assignee: University of Cincinnati, Cincinnati, OH (US)

(21) Appl. No.: 19/088,210

(22) Filed: Mar. 24, 2025

Related U.S. Application Data
(60) Provisional application No. 63/568,785, filed on Mar. 22, 2024.

(51) Int. Cl. A61B 18/14; A61B 18/00

(52) U.S. Cl. CPC A61B 18/148; A61B 2018/00446; A61B 2018/00577; A61B 2018/00791

(57) ABSTRACT

A method of performing radiofrequency lesioning on a subject is provided. The method involves introducing an outer cannula and an inner cannula into the subject percutaneously, then guiding the outer cannula and inner cannula using neuronavigation, where the inner cannula is neuronavigation-compatible and the neuronavigation uses a navigated posterior clival line (nPCL) as an anatomic reference point. Then, removing the inner cannula from the outer cannula once the outer cannula and inner cannula are placed in a position for creating a lesion in the subject. Next, inserting an electrode into the outer cannula; and passing an electrical impulse through the electrode, causing a lesion in the subject.

### CROSS-REFERENCE TO RELATED APPLICATIONS

This application claims priority to U.S. Provisional Application Ser. No. 63/568,785, filed Mar. 22, 2024, which application is hereby incorporated by reference in its entirety.

### TECHNICAL FIELD

The present invention relates to radiofrequency lesioning techniques.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Radiofrequency lesioning (RFL) is a safe and effective treatment modality for medically-refractory trigeminal neuralgia (TN). Due in large part to the work of Dr. John Tew, RFL gained mainstream neurosurgical acceptance in the 1970s. However, the technique has remained relatively unchanged, relying on lateral fluoroscopy.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, a method of performing radiofrequency lesioning on a subject in need of such treatment is provided. The method involves introducing an outer cannula and an inner cannula into the subject percutaneously, then guiding the outer cannula and inner cannula using neuronavigation, where the inner cannula is neuronavigation-compatible and the neuronavigation uses a navigated posterior clival line (nPCL) as an anatomic reference point. Then, removing the inner cannula from the outer cannula once the outer cannula and inner cannula are placed in a position for creating a lesion in the subject. Next, inserting an electrode into the outer cannula; and passing an electrical impulse through the electrode, causing a lesion in the subject.

In one embodiment, the radiofrequency lesioning is performed without the use of lateral fluoroscopy. In another embodiment, the inner cannula is connected to an L-shaped probe. In one embodiment, the inner cannula is straight. In another embodiment, the inner cannula has a curve.

In another aspect of the present invention, a radiofrequency lesioning kit is provided. The kit has an outer cannula, an L-shaped extended probe connected to an inner cannula, an electrode capable of passing through the outer cannula and a power source connected to the electrode. In one embodiment, the inner cannula is neuronavigation-compatible.

### BRIEF DESCRIPTION OF THE DRAWINGS

The objects and advantages of the disclosed invention will be further appreciated in light of the following detailed descriptions and drawings in which:

FIG. 1 is a schematic showing a current TEW kit.

FIG. 2 is a schematic showing a CAD model of a TEW kit.

FIG. 3 is a schematic showing a laser scanning device.

FIG. 4 is a schematic showing an embodiment of the novel TEW kit of the present invention.

FIG. 5 is a schematic showing a 3D printed stainless steel TEW kit according to the present invention.

FIG. 6 is a schematic showing a 3D printed stainless steel TEW kit according to the present invention.

FIG. 7 is a photo showing an embodiment of the inner cannula of the present invention.

FIG. 8 is an image showing different outer cannula-inner cannula combinations.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Radiofrequency lesioning (aka ablation) is a pain management treatment that disrupts nerve signals on a long-term basis. It is a minimally invasive procedure that uses radiofrequency energy (electrical impulses) to destroy damaged nerves or tissues that send pain signals to the brain. By disrupting the communication between the nerve and the brain, the patient's pain is reduced. This outpatient procedure can provide lasting relief in patients with chronic pain. Radiofrequency lesioning is typically recommended for patients who have not found relief from other interventional pain treatments such as physical therapy or nerve blocks.

RFL is currently performed using a TEW kit under fluoroscopic guidance (X-Ray) to assist in accurate needle placement to the medial nerves that are being treated. With the use of a radiofrequency needle and a microelectrode, the site and surrounding tissues are stimulated by a small radiofrequency current. The X-Ray guidance allows the doctor to watch in real-time to make sure the needle is being injected into the correct location. Occasionally, the doctor may use contact to confirm the correct needle placement. Patients have reported slight discomfort, but usually feel more pressure than pain. The electrical impulse is passed through the needle and creates a small burn, called a lesion. This heats the nerve to approximately 80 degrees Celsius, which then destroys the nerve, blocking the pain signals. Multiple nerves can be burned at the same time. If successful, the procedure can be done multiple times.

A TEW kit is used for radiofrequency lesioning procedures such as percutaneous RF lesion-making in the trigeminal ganglion for the treatment of trigeminal neuralgia. A TEW kit may use either off-axis or straight electrode tip extensions for flexibility in lesion size and positioning in the ganglion. In one embodiment, the kit includes an insulated 19 gauge cannula into which a straight electrode or a curved tip electrode can be inserted. For example, a straight electrode is inserted in the cannula if an axial tip extension is desired. Alternatively, a curved electrode is inserted into the cannula if an off-axis tip extension is needed to reach difficult positions, such as the V1 ganglion division. The electrodes have thermocouple temperature sensors built into their tips. The TEW kit uses a cable to connect the electrodes to a lesion generator.

Radiofrequency lesioning can be used to treat chronic pain conditions such as facet joint nerves, sacroiliac joint nerves, peripheral nerves, spinal arthritis and stenosis, chronic back pain, sprains and strains. An advantage of RFL is that it works and blocks the pain signals to provide long-term relief. Patients who have success with radiofrequency lesioning can expect relief for 6 to 9 months.

Neuronavigation is a set of computer-assisted technologies used by neurosurgeons to guide or “navigate” within the confines of the skull or vertebral column during surgery. The present invention uses neuronavigation in lieu of lateral fluoroscopy for selective targeting of trigeminal rootlets during radiofrequency lesioning. The inventive process allows for more selectivity than lateral fluoroscopy. In one embodiment, the present invention is a step towards a fully neuronavigation-compatible RFL system, expanding upon and modernizing Dr. Tew's pioneering work. The neuronavigation of the present invention allows for higher selectivity when performed for RFL. As used herein, “neuronavigation-compatible” means a device that works in cooperation with neuronavigation computer-assisted technologies to aid in the neuronavigation process.

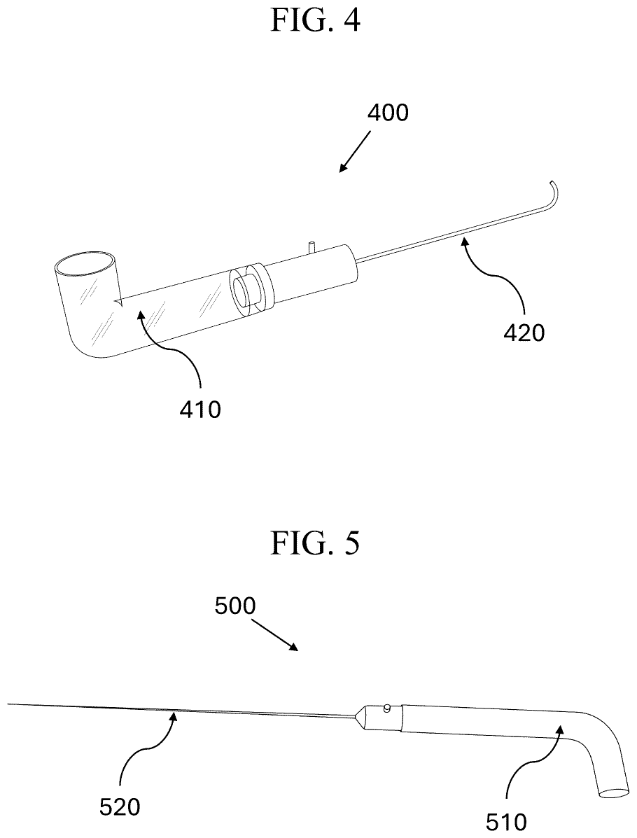

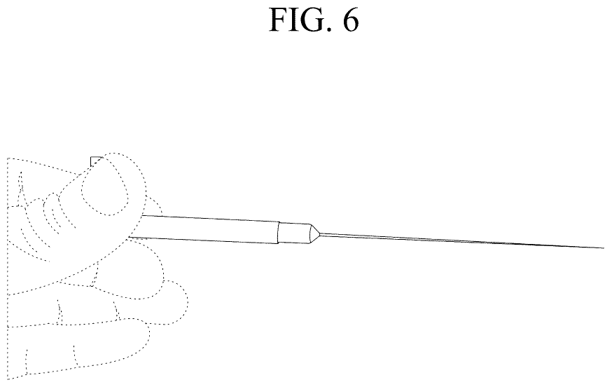

A neuronavigation-compatible system for RFL was designed using a novel RFL kit. Referring to FIG. 4, the novel RFL kit 400 has an extended probe 410 similar to an L-bracket structure, allowing the user to control the RFL kit in the orthogonal direction (see FIGS. 5 and 6). An alternate embodiment of the novel RFL kit of the present invention 500 shows an extended probe 510 with a straight inner cannula 520, as opposed to the curved inner cannula 420 of RFL kit 400.

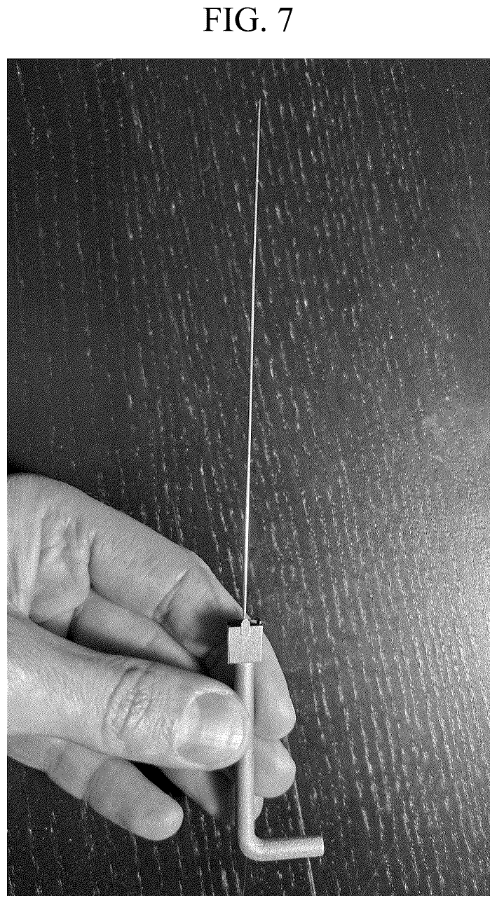

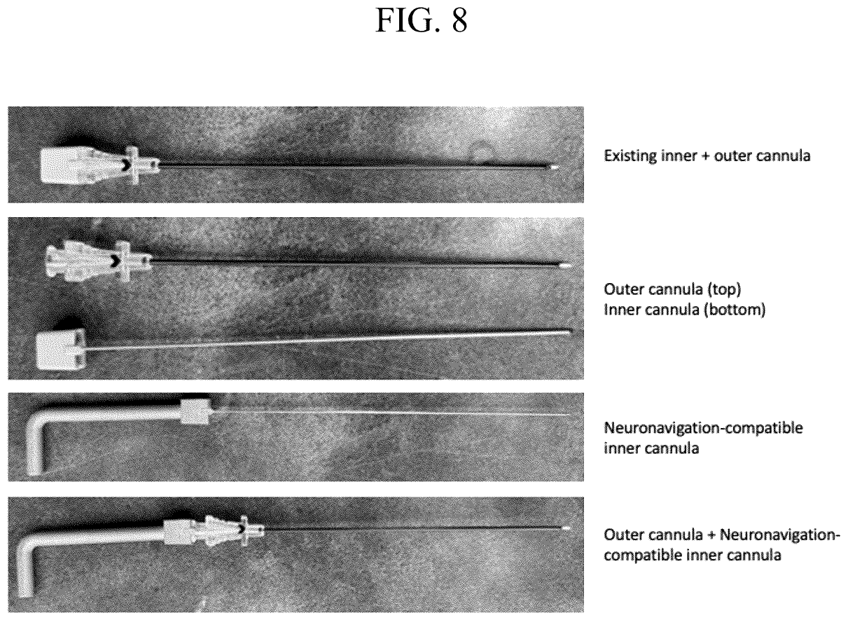

The radiofrequency needle of the present invention comprises an inner cannula and an outer cannula (see FIGS. 7 and 8). Both the inner and outer cannulas go in together. The inner cannula is configured in such a manner to support neuronavigation. The inner cannula is then removed and an electrode is threaded through the outer cannula to the desired target.

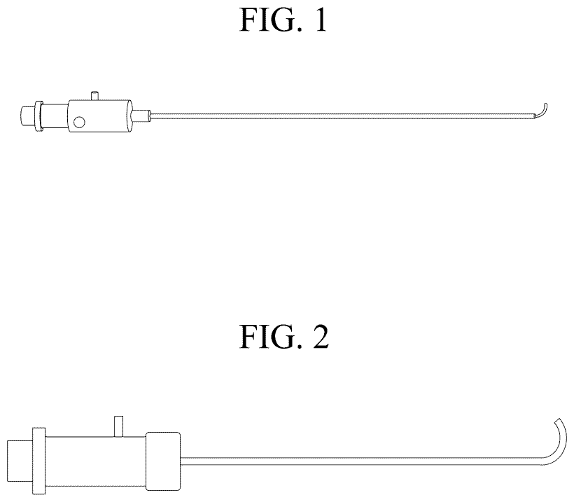

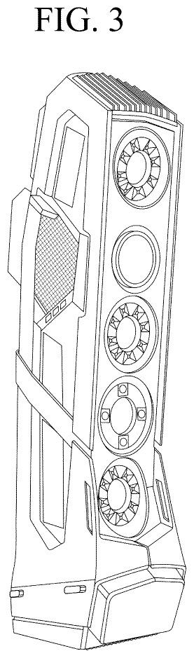

The novel RFL kit was generated by taking a 3D scan of a currently available prosthetic (FIG. 1) using a laser scanner (FIG. 3) to generate a CAD model (FIG. 2) that remains accurate and precise. In one embodiment, the novel RFL kit and the extended probe are manufacturable through Additive Manufacturing (AM) (3-D printing) methods. This was confirmed by checking it with our custom “Design for Additive Manufacturing” (DfAM) algorithm and apps and appropriately modifying the design.

The present invention uses neuronavigation and a navigated posterior clival line (nPCL) as an anatomic reference point. This approach allows the invention to contact each trigeminal rootlet with high rates of success. The algorithm used for selective targeting was slightly modified from that which was originally described by Dr. Tew. This improved algorithm is better suited for use with neuronavigation. Our morphometric data, particularly the distance from the foramen ovale (FO) to the nPCL was highly uniform between specimens, indicating its utility as a key parameter. Our results indicate that incorporating neuronavigation into RFL should improve operative efficiency, accuracy, and ultimately improve outcomes.

### EXAMPLES

### Example 1

Twenty RFL procedures were performed on embalmed cadaveric specimens. After pre-procedural thin-cut CT scans were obtained, specimens were registered to neuronavigation and frontotemporal craniotomy was performed to facilitate direct visualization of the Gasserian ganglion. Pre-planned trajectories were created using an “entry” 2.5 cm lateral to oral commissure and “target” at FO. A 19-gauge TEW cannula was retrofit to the modified end of a navigation probe, adjusting the “offset” function to permit real-time tracking. Using Hartel's technique, the cannula was advanced through FO to the navigated posterior clival line (nPCL). A curved TEW electrode was inserted and oriented inferolaterally for V3 and superomedially for V2. For V1, the cannula was advanced 5 mm beyond the nPCL and the curved electrode was oriented inferomedially. At each position, a surgical microscope was used to evaluate whether successful contact was achieved. Relevant distances and trajectory angles were measured and recorded.

Successful contact with V3, V2, and V1 was made in 95%, 90%, and 85% of attempts, respectively. The mean distance from entry point to FO was 7.61 cm±0.74 cm. The mean distance from FO to the nPCL was 1.26 cm±0.25 cm. The mean coronal and sagittal trajectory angles were 22.8°±6.6° and 50.6°±6.2°, respectively.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method of performing radiofrequency lesioning on a subject in need of such treatment, the method comprising:
a. introducing an outer cannula and an inner cannula into the subject percutaneously;
b. guiding the outer cannula and inner cannula using neuronavigation, wherein the inner cannula is neuronavigation-compatible and the neuronavigation uses a navigated posterior clival line (nPCL) as an anatomic reference point;
c. removing the inner cannula from the outer cannula once the outer cannula and inner cannula are placed in a position for creating a lesion in the subject;
d. inserting an electrode into the outer cannula; and
e. passing an electrical impulse through the electrode, causing a lesion in the subject.

2. The method of claim 1 wherein the radiofrequency lesioning is performed without the use of lateral fluoroscopy.

3. The method of claim 1 wherein the inner cannula is connected to an L-shaped probe.

4. The method of claim 1 wherein the inner cannula is straight.

5. The method of claim 1 wherein the inner cannula comprises a curve.

6. A radiofrequency lesioning kit comprising an outer cannula, an L-shaped extended probe connected to an inner cannula, an electrode capable of passing through the outer cannula and a power source connected to the electrode.

7. The radiofrequency lesioning kit of claim 6 wherein the inner cannula is neuronavigation-compatible.
